(19) United States
(12) Patent Application Publication
(10) Pub. No.: US 2008/0057497 A1
(43) Pub. Date: Mar. 6, 2008

(54) BIOLUMINESCENCE RESONANCE ENERGY TRANSFER REPORT ELEMENT FOR BIOLOGICAL ARRAYS

(76) Inventors: Brian John Filanoski, Sonkane, WA (US); Eric Cameron, Coeur D'Alene, ID (US); Wusi Maki, Coeur D'Alene, ID (US)

Correspondence Address: TECHNOLOGY LAW GROUP, LLC, 8950 W. EMERALD STREET STE. 198, BOISE, ID, 83704, US

(21) Appl. No.: 11/469,288

(22) Filed: Aug. 31, 2006

(51) Int. Cl. C12Q 1/68; C12Q 1/66

(52) U.S. Cl. 435/6; 435/8

(57) ABSTRACT

The present invention provides methods for the detection of bioluminescence resonance energy transfer. The present invention provides methods of generating a report element and its applications in high throughput detection of bio-agents and gene expression levels. The present invention also relates to a nucleic acid signal amplification process.

The present invention provides a report element where a fluorescent dye is directly conjugated to redox enzyme for use in bioluminescence resonant energy transfer (BRET) assays. The present invention also provides a pair of highly homogeneous luciferases for a dual BRET report system allowing the comparison of gene expression levels. With this report element, charge coupled device (CCD) and CMOS based optical detection mechanisms may be used for highly sensitive, high throughput and low cost portable luminescence biosensors.

A nucleic acid based signal amplification process is provided for a universal protein detection platform allowing the replacement of protein chips while providing a more stable platform.

### GOVERNMENT SUPPORT

This invention was funded in part by USDA Grant No. 344791654 and NASA Grant No. NNG05GC51G. The United States Government has certain rights in the invention.

### BACKGROUND OF THE INVENTION

1. Field of Invention

The present invention relates to a bioluminescence resonance energy transfer (BRET) method for reporting the presence of biological agents. The present invention relates to the development of single and dual BRET report systems for the comparison of gene expression levels and other bio-detection. The present invention also relates to a nucleic acid signal amplification process for a universal protein detection platform. The present invention also relates to analysis software for the differentiation and quantification of light signals from a bioluminescence reaction and resonance energy transfers.

2. Background

Currently research into biosensor technologies focuses mainly on bio-agent detection. Numerous new technologies have been developed recently such as optical biosensors using fluorescence based assays. These assays, in addition to using the traditional direct and indirect labeling of a biomolecule with fluorescent dyes, have recently implemented quantum dots as new fluorescent elements. The currently commercially available DNA and protein microarrays are mostly based on fluorescent technology.

Fluorescent assays are very sensitive and this great sensitivity may present problems when bio-agents are assayed. This is because many bio-agents have some inherent fluorescence properties which may interfere with fluorescence assays. Auto-fluorescence is the most common problem in fluorescent detection. This auto-fluorescence must be accounted for when running an assay in order to obtain accurate results. Reports indicate that five percent or more of biological materials have fluorescent properties that may interfere in any analysis using fluorescent probes. The result of such inadvertent fluorescent properties is higher background noise that interferes with the analysis and leads to decreased sensitivity and specificity. Another problem with fluorescent assays is photo-bleaching which occurs when the fluorescent molecule no longer responses or responses poorly when exposed to it excitation light. Finally, fluorescent detection systems require an expensive light source coupled to an expensive detection system in order for the excited optical element to be detected.

Bio-luminescent assays are exquisitely sensitive and are not limited by overlap between the fluorescent properties of analytes and assay components. Bio-luminescent detection when combined with novel surface modification technologies can achieve high sensitivity and low background noise. Thus, bioluminescent assays may be use in the detection of low concentration target agents in vivo. Bioluminescent assays have been reported by several researchers using an in vitro application on an optical chip-based detection platform. The optical chip-based detection platform has showed great potential towards the development of a highly sensitive and low cost biosensor. However, since bioluminescence detection is based on enzymatic reaction, the diffusion of reaction products, from which are generated the light signal, limits its applications in high throughput detection (array) platforms, because these high throughput detection platforms (or arrays) generally require a high spatial resolution.

Bioluminescence resonance energy transfer technology (BRET) is similar to fluorescence resonance energy transfer (FRET), in that both are powerful tools for reporting molecular interaction events. BRET technology takes advantage of fact that the energy donors are the products of bioluminescent reaction (i.e. enzymatic reduction), and the energy acceptors are fluorescent proteins. A comparison of FRET vs. BRET shows that the analysis of FRET signals are more complicated due to fluorescent bleed-through, auto-fluorescence as well as photo-bleaching. BRET has the advantage of producing a simple meaningful analysis from simple apparatus. However, both technologies depend upon the efficiency of energy transfer. This energy transfer is extremely sensitive to the distance between donor and acceptor. Thus, there is a need for a new report element that will minimize the distance between donor and acceptor as a way to increase the sensitivity of the analysis.

Bioluminescence detection is based on enzymatic redox reaction to generate light activity, thus the speed of turnover and substrates of different enzymes presents problems in this system. This difference marks it difficult to compare gene expression level when using two different enzymes as reporters. Current technologies in gene expression arrays are all based on fluorescence methods.

Recently Promega®, has developed the Chroma-Luc report system. Chroma-Luc report system is a plasmid vector based system. The Chroma-Luc report system is currently used for in vivo study through vector transfection. The Chroma-Luc report system contains both red-emitting and green-emitting luciferases which were derived from the native yellow-green luciferase gene originally cloned from a large click beetle. These two reporter enzymes are highly homologous in sequence structures (>98% amino acid identity) and in their activity behavior. In contrast to the firefly luciferase activity, the life time of these two enzymes is much longer. This longer enzyme life time makes these two enzymes better reporter enzymes for use in Charge Coupled Device (CCD) based detection. In addition, signals may be generated in the presence of the same substrate for both enzymes.

The light detectors in luminometers are photomultiplier (PMT) based. The PMT efficiency of a luminometer is more sensitive in the blue and green wavelengths and least sensitive in the red wavelength. Since light generated from BRET are in the relatively long wavelength region, CCD detectors are more sensitive in the detection of signals, as the CCD is more sensitive in longer wavelength region, particularly in red spectrum. CCD not only works well for detecting BRET signal, can also be a device model for developing optical chip-based biosensors, i.e. CMOS chip-based protein arrays.

Proteins on a biochip have a short shelf-life, since their biological function is unstable when they are immobilized on a solid surface. Nucleic acids and short peptide nucleic acids on solid surface are much stable compared with proteins, since they can be store in dry condition. An alternative protein detection method has been developed to convert protein detection into nucleic acid detection.

Thus, there exists a need for a bioluminescence assay system which lacks fluorescent bleed-through, that is not affected by auto-fluorescence of a bio-agent and is not subject to photo-bleaching. Such a system would result in a simple analysis using simple apparatus. Additionally, there exists a need for a bioluminescence assay for the comparison of gene or protein expression levels that is both sensitive yet relatively simple. Finally, there exists a need for a biochip with an extended shelf-life.

### SUMMARY OF THE INVENTION

Accordingly, the present invention is directed to bioluminescence resonance energy transfer methods for detecting bio-agents and comparing gene/protein expression levels on CCD or CMOS portable detector.

Additional features and advantages of the invention will be set forth in the description which follows, and in the part will be apparent from the description, or may be learned by practice of the invention. The objectives and other advantages of the invention will be realized and attained by the methods particularly pointed out in the written description and claims hereof as well as the appended drawings.

To achieve these and other advantages and in accordance with the purpose of the invention, as embodied and broadly described, the invention relates to a method for the detection of bio-agents in biological samples using a bioluminescence resonance energy transfer (BRET) assay comprising the steps of obtaining a biosample; contacting the biosample with a report element; allowing the report element to capture its target bio-agent; adding substrate for the report element; generating a light signal through BRET; detecting the light signal with a sensor.

In another aspect, the invention includes a method for the comparison of bioagent levels in biological samples using a BRET assay comprising the steps of obtaining two different biological samples; contacting a pair of report elements with both samples to capture targets; contacting the report elements with substrate; generating two light signals through BRET; detecting both light signals. Preferably, the report element comprises a pair of fluorescent elements conjugated to a redox enzyme. More preferably, the report element comprises a pair of homologous redox enzymes. Even more preferably, the pair of homologous redox enzymes comprise Luciferase-red and Luciferase-green. The two report elements are linked to two anti-tag antibodies. Two tags are used to label bioagents from the two samples. Specific recognition elements are used to capture target from two samples. The interaction of the tags and their antibodies generates two light signals when substrate is present. The recognition elements may be antibodies, receptors, ligands, nucleic acids or other biomolecules.

In yet another aspect, the invention includes a method of signal amplification using BRET assay comprising a recognition element; a signal molecule; peptide nucleic acids immobilized on a detection surface; a report element; generation of a light signal through BRET; detection of the light signal. The optical sensor is selected from the group consisting of CCD based detectors, CMOS based detectors, luminometers, or light sensitive films.

In another aspect, the invention includes a dual BRET report system.

In another aspect, the invention includes an alternative protein detection method using nucleic acid and/or peptide nucleic acid detection.

In another aspect, the invention includes a reagent kit for the detection of bio-agents in a sample.

It is to be understood that both the foregoing general description and the following detailed description are exemplary and explanatory and are intended to provide further explanation of the invention as claimed.

The accompanying drawings are included to provide a further understanding of the invention and are incorporated in and constitute a part of this specification. The accompanying drawings illustrate several embodiments of the invention and together with the description serve to explain the principles of the invention.

### BRIEF DESCRIPTION OF THE DRAWINGS

FIG. 1 is a schematic diagram depicting the structure of report element.

FIG. 2 is a schematic diagram depicting the detection of bio-agents through the use of bioluminescence resonant energy transfer.

FIG. 3 is a schematic diagram depicting spatial resolution of bioluminescence resonance energy transfer.

FIG. 4 is a schematic diagram depicting the comparison of gene expression by dual BRET report system.

FIG. 5 is a schematic diagram depicting the lights differentiation.

FIG. 6 is a schematic diagram depicting a universal detection platform.

FIG. 7 is a comparison of light intensities of using different substrates for alkaline phosphatase dual system.

FIG. 8 is a schematic diagram depicting the CCD or CMOS portable detector.

### DETAILED DESCRIPTION

The present invention is directed to bioluminescence resonance energy transfer methods for detecting bio-agents and comparing gene/protein expression levels on CCD or CMOS portable detector.

In general, energy donors in BRET are products of bioluminescent reaction, and energy acceptors are fluorescent proteins. The present invention describes a report element in which fluorescent dyes are directly conjugated to enzymes that catalyze a redox reaction. Previously it has been reported that attempts to directly couple fluorescent dyes to enzymes has resulted in either the lost or greatly reduced enzyme activity. This lost or reduction of activity may have been due to steric hindrance conformational changes in the enzyme's active site. Surprisingly the present invention shows no such lost of enzymatic activity.

Additionally, the present invention solves another problem associated with process of bioluminescence resonance energy transfer assays when used in high throughput arrays that being a low spatial resolution. The present invention has reduced the transfer distance between donor and acceptor resulting in higher transfer efficiency. energy thus yielding a higher spatial resolution. Thus, the present invention achieves a better spatial resolution and may be use in high throughput arrays.

In an embodiment of the present invention, bio-agents in a biological sample are detected using a bioluminescence resonance energy transfer (BRET) assay. In this embodiment, redox enzymes conjugated to fluorescent elements are employed as report elements. Redox enzymes include but are not limited to alkaline phosphatase, luciferases, and horse radish peroxidase. One of ordinary skill in the art will readily recognize that any other redox enzyme may be substituted in this method. Similarly, the fluorescent elements include but are not limited to fluorescent dyes, fluorescent proteins and quantum dots. The fluorescent elements may be directly or indirectly linked to the redox enzyme.

In this embodiment, the bioluminescence resonance energy transfer refers to the light generated from enzyme redox reaction that is absorbed by the fluorescent element. The fluorescent element emits the second light with a longer wavelength. The analysis begins by obtaining a biological sample. The recognition element is applied to biological sample thereby allowing the capture or interaction of the recognition element specific target bio-agent which may or may not be present in the biological sample. The recognition elements maybe antibodies, receptor, ligands, nucleic acids, or other bio-molecules.

The presence of the bio-agent is detected by the application of the substrate for the enzyme redox reaction. The substrate-enzyme redox reaction generates a light signal from the fluorescent molecule. The energy from the light signal is passed along the fluorescent molecules and generates the second light signal through the bioluminescence resonant energy transfer where it the second light signal is detected by an optical or non-optical (could be electrical) sensor. The optical sensors may be CCD or CMOS based detectors, luminometers, films, or other sensing mechanisms.

For the comparison of gene expression levels in two samples, a pair of highly homogeneous luciferases is used as reporters. For target detection and quantification, two signal amplification processes and one negative detection approach are presented. Lumino-enzyme is used to report the existence of target agents or signal molecules.

In this embodiment of the invention a pair of fluorescent elements is linked to a pair of highly homologous redox enzymes to generate two report elements. The two report elements are linked to two anti-tag antibodies. The two tags are used to label biological agents from two samples through the specific recognition elements used to capture targets from both samples. The interaction of tags and their antibodies results in the generation of two light signals from the report elements, which in the presence of their substrate generates two light signals. The two light signals are emitted from the two fluorescent elements via bioluminescence energy transfer and are detected in an optical sensor.

Another embodiment of the invention involves a pair of highly homogeneous luciferases, one red-emitting and one green emitting. Gene expression level in two samples can be compared by using two tags, two anti-tag antibodies and two luciferases to generate two light signals. In this embodiment of the present invention a pair of genetically modified and highly homogenous luciferases is used to report and compare gene expression level in two samples in vitro. Target agents in two samples are labeled with two different tags. Target agents are captured on the detection surface by their specific recognition element. Luciferases previously conjugated with anti-tag antibodies are introduced to the detection surface through the interaction between tag and its antibody. Two light signals are generated by the two luciferases. For the detection and quantification of the amplified light signal, a portable, charge coupled electronic sensor device is designed. A disposable detection platform has been designed as the reaction chamber. An integrated microprocessor analyzes the light signals relative to the gene expression levels.

In another embodiment of the invention a signal amplification process disclosed. In this embodiment an oligonucleotide is linked to a recognition element. The specific sequences of the oligonucleotide are the target for the recognition element. The sequence of oligonucleotide consists of tandem repeats. Biotin labeled complimentary repeats hybridized to the oligonucleotide serve as signal molecules. The complimentary repeats are released from oligonucleotide when the recognition element binds to the target. Repeats are captured by peptide nucleic acids that are immobilized on a detection surface. The conjugate of streptavidin-report element binds to the nucleic acid repeats. A light signal is generated from fluorescent element through bioluminescence resonant energy transfer and the light/signal is detected by an optical or non-optical (could be electrical) sensor.

Nucleic acid barcode represents the presence of specific protein was used as detection signals. Complimentary sequence of barcode is designed as specific recognition element to capture signal molecules on a solid surface. This nucleic acid or peptide nucleic acid detection platform not only has long shelf-life, also is a universal detection platform, since the same barcode can be used to present different protein target in different detection events.

Charge coupled device has been used for studying detection models. This BRET based detection mechanism has broad applications in molecular detection both in vitro and in vivo. More importantly, it may take place in a high throughput assay.

In another embodiment of the invention a signal amplification system utilizes a conjugate of antibody-oligonucleotide tendon repeats. Each repeat presents a signal molecule.

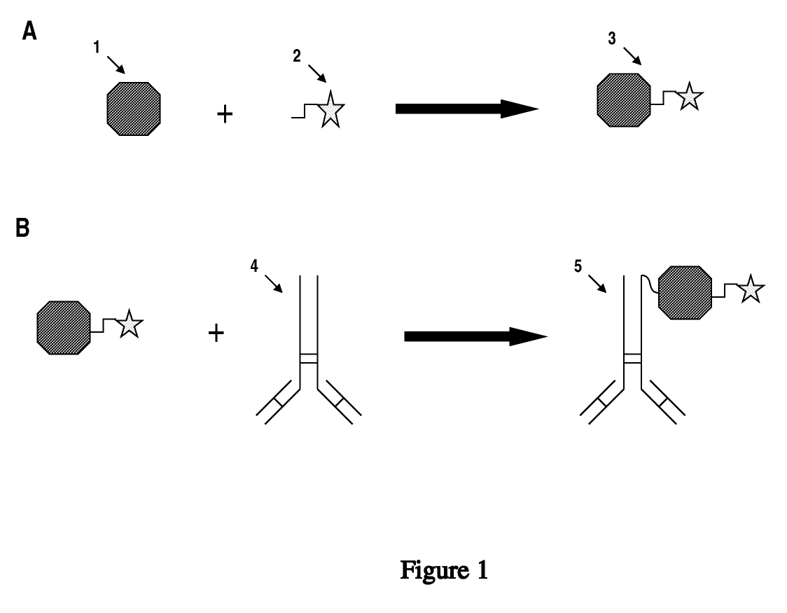

Referring now to figure to FIGS. 1(A) and (B), where the constructions of report element and its antibody conjugate are shown. In FIG. 1(A) the Redox enzyme (1) reacts with fluorescent dye hydroxysuccinimide (NHS) ester (2) to form a report element (3). In FIG. 1(B) the Report element (3) is linked to a recognition element, i.e. antibody (4) to form a conjugate (5) of antibody and report element, through a chemical conjugation procedure, or affinity binding, i.e. His-tag/nickel metal interaction as described in Example 3.

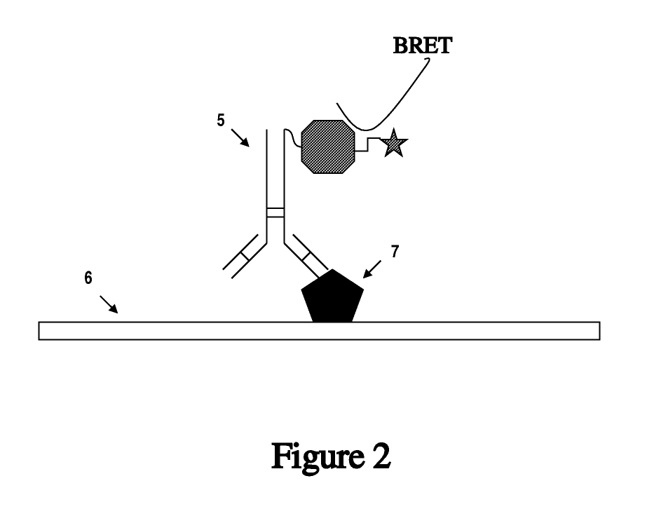

Referring to FIG. 2, where the detection mechanism of present invention is shown. Target bio-agent (7) is captured on solid surface (6) directly or indirectly. The conjugate (5) of target specific recognition element and report element binds to the bio-agent. Fluorescent light signal will be generated in the presence of a substrate of redox enzyme through BRET.

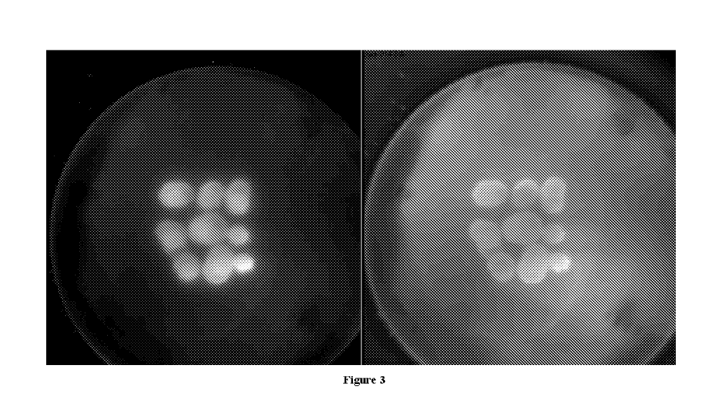

Referring to FIGS. 3(A) and (B), where the spatial resolution of BRET is shown. The conjugate of alkaline phosphatase-AF555 (Molecular Probes) was spotted on a 1 cm² membrane. Substrate Atto-Glow530 was used to generate 530 nm light in the presence of alkaline phosphatase. FIG. 3(A) is a photograph showing the results when a filter HQ560L is used to filter out 530 nm light. The light signal was from AF555 through BRET. FIG. 3(B) is a photograph showing the results without filter. Lights signals were generated from both atto-Glow530 and AF555.

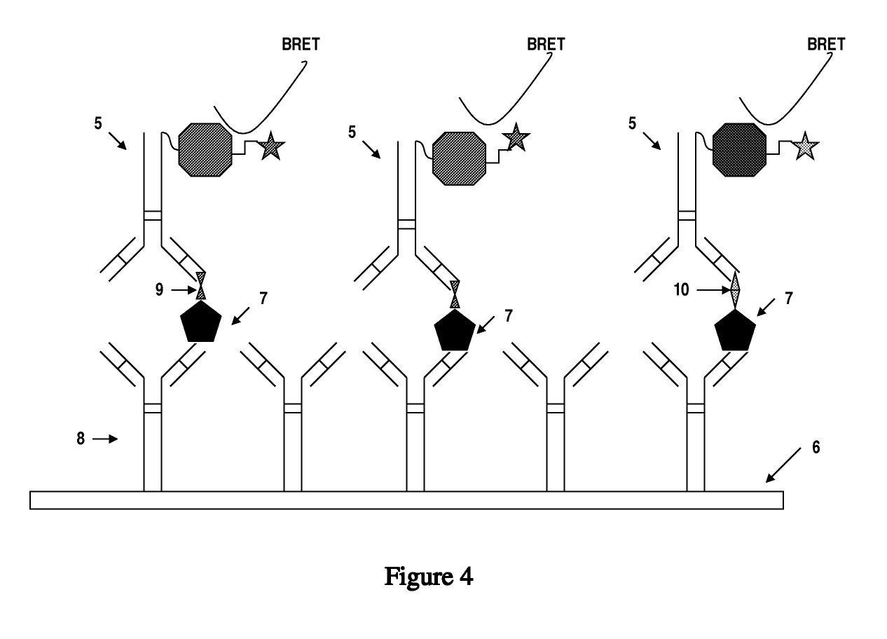

Referring to FIG. 4, where the detection mechanism is shown. Target bio-agents (7) from two samples are labeled with different tags (9) and (10). Specific antibodies (8) capture their target agent on a solid surface (6). Conjugates of anti-tag antibody-report element interact with tag and generate light signal through BRET.

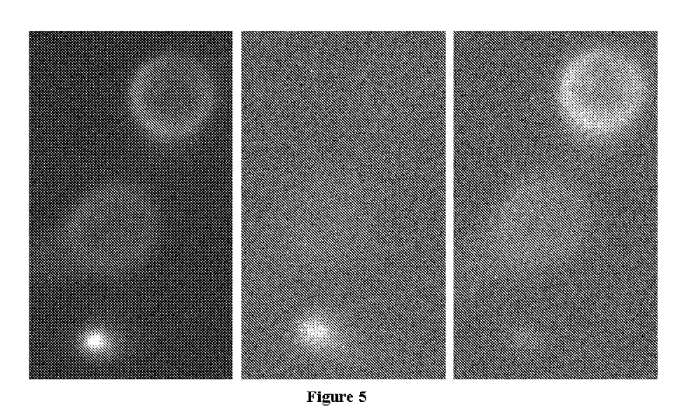

Referring to FIG. 5, where the light differentiation from two luciferases is shown. Luciferases were spotted on a membrane in the flowing order: luciferase-red, a two luciferase mixture and luciferase-green, from top to bottom. Substrate Chroma-Luc was used to generate light signals, 537 nm for green and 613 nm for red. Photographs were taken as follows from left to right: without filter, with a green-pass filter, and with a red-pass filter.

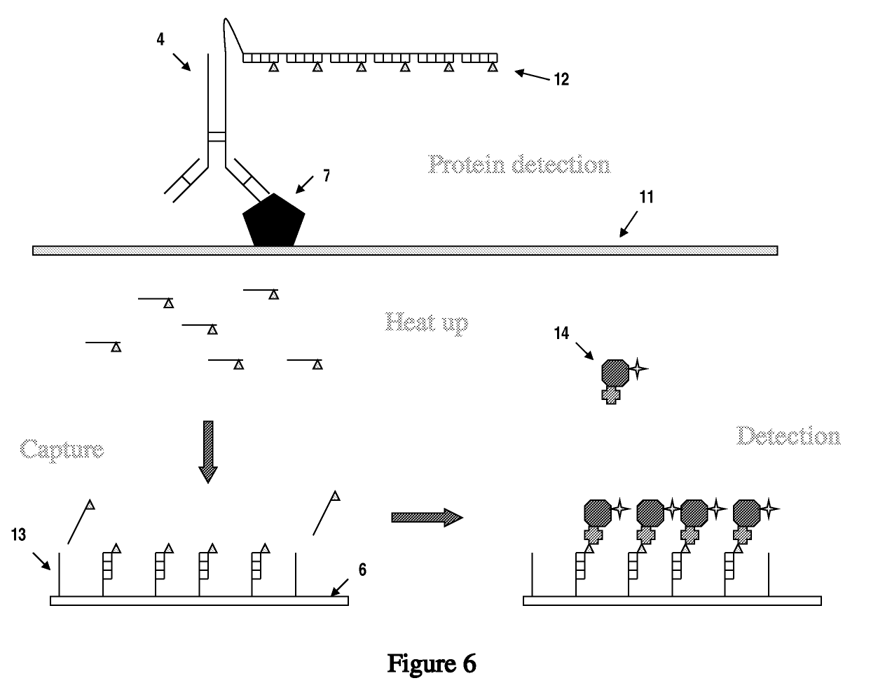

Referring to FIG. 6, where an alternative detection mechanism is shown. Bio-agent specific antibody (4) linked to an oligonucleotide with tendon repeat sequence (12). This DNA-antibody conjugate was used to detect target bio-agent. Tag-repeats (12) were released from the conjugate as signal molecules. Tag-repeats (12) were captured through hybridization with peptide nucleic acids (13) immobilized on a detection surface (6). Conjugates of report element and a tag-binding molecule (14) attach to the hybrids through the interaction of tag and tag-binding molecule. Light signal was generated through the BRET process.

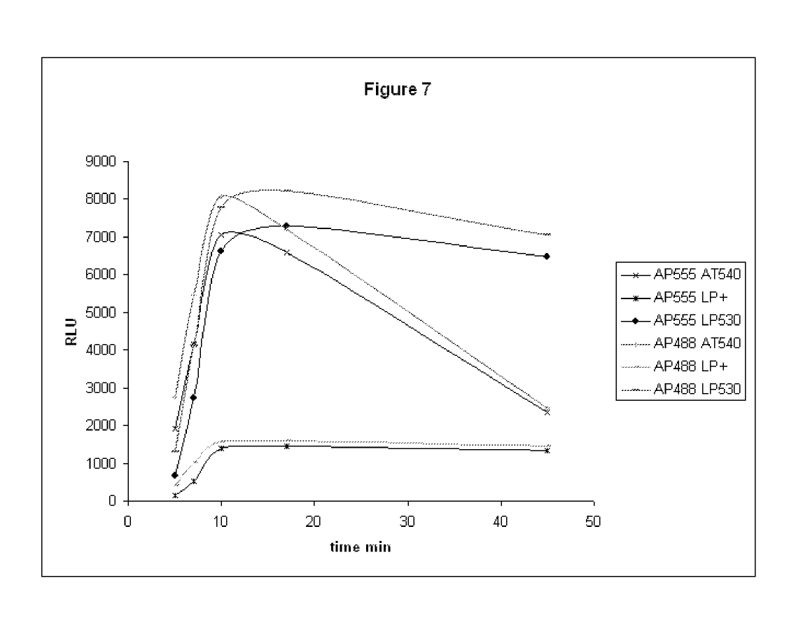

Referring to FIG. 7, alkaline phophatase (AP) report elements were tested by using different substrates. One report element is AP-AF555 conjugate (red lines) which contains average 5 fluorescent dyes per enzyme. The other is AP-Alex Fluor 488 (black lines) which contains average 4.5 fluorescent dyes per enzyme. Three substrates were used to test BRET. Lumi-phos Plus (425 nm) showed least efficiency of BRET, Lumi-phos (530 nm) showed much higher efficiency, but signal reduced quickly. AttoGlow (540 nm) showed high efficiency and signal that lasted much longer.

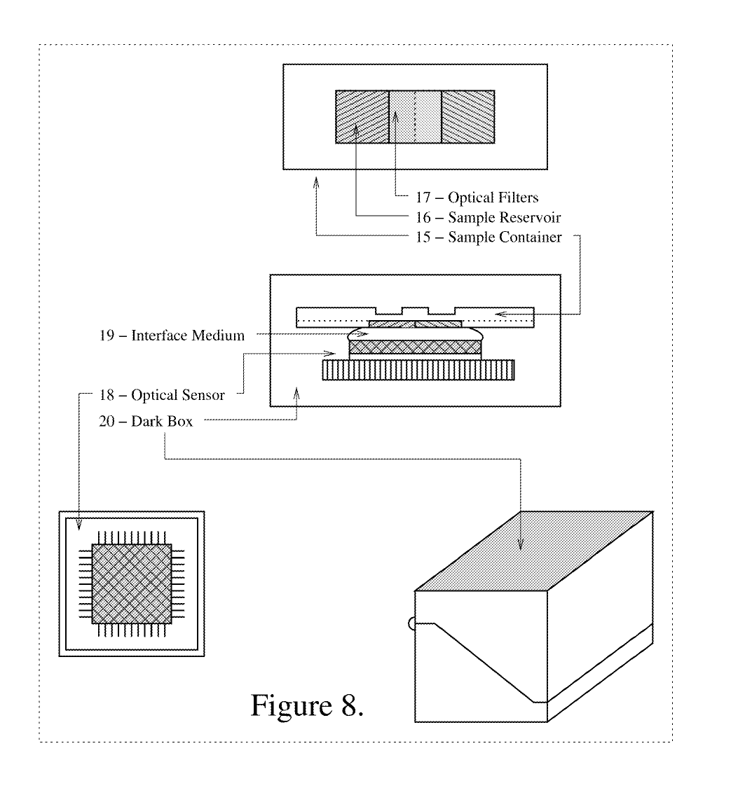

Referring to FIG. 8, where a basic block diagram of the CCD portable detector is shown. The CCD portable detector consists of three major components. The first component of the CCD portable detector is the sample container (15). The first component comprises two sub-parts. The first sub-part is the sample reservoir. For this, several configurations are possible, and there may be multiple reservoirs. Each reservoir is holds a sample. An example reservoir configuration is given as reference(16). In this example, the container is a flat plane of glass in with two etched micro-channels. There is a central un-etched region between the micro channels.

The second sub-part of the sample container is an optical filter assembly (17). This provides for any optical corrections that may be required to insure good signal transfer. The optical filter assembly may, for example, provide for optical signal focusing and/or wavelength separation. Given that many variations are possible for sample container construction; several important factors to be considered are that the reservoirs effectively contain the samples, and that the optical filter assembly effectively conveys the optical signal toward the second component of the device, the optical sensor (18). The sample container is placed at the detection surface of the optical sensor. An interface medium (19) may be used to insure good signal transfer to the optical sensor. This medium acts in conjunction with the optical filter assembly.

The second major component of the detection device is the optical sensor (18). For this component, one of several commercially available image detection elements will may be used These are well known in the art and a person of ordinary skill in the art can readily and easily determine the best suited element. A commercially available charge coupled device (CCD) image sensor is preferred. For this reason, the entire electronic detector shall be referred to as the CCD portable detector. The optical sensor directly detects and digitizes the amplified signal. The sensor can simultaneously detect multiple signals.

The digitized signal is then transferred to the third component of the device. This third component of the device is the digital signal analyzer. The preferred mode is to generate a software program capably of operating on a personal computer. The third component accepts the digitized signal from the optical sensor, corrects the signal for optical noise and sensor aberrations. It then quantifies the signal received into the actual concentration of the signal molecule or, in the multiple sample case, molecules. The entire instrument is enclosed in a case (20) that prevents light or other contaminants from disturbing the detection process.

The differentiation and detection software accepts input from the CCD or other technology based photo-detection array in a standard format that is independent of the exact photo-array used. Through this mechanism, it has the capability to analyze results from a large number of different sensor arrays. It should be noted that, in the typical case, data for at least one, and often multiple samples, will be captured for a single analysis. Moreover, multiple elements of the sensor array are typically used to gather data for each sample. The software, with the aid of input signal calibration data, automatically determines the number of sensor array elements used to gather data for each sample, and also automatically determines the number of samples. With the aid of sensor calibration data, the software normalizes the input data to account for variations in sensor sensitivity across the array. The software also analyzes and digitally filters the input data to minimize the effects of spurious signal recorded that is unrelated to the light captured from the input samples, i.e. sensor noise. The software then calculates the amount of light detected from each sample. If the sensor input data contains color information, the software calculates amount of light of each of the detected colors. Further analysis requires specific information about the input samples. This information includes such elements as which samples are control samples, what, if any, filtration or diffraction was used during data gathering for individual/specific samples, which samples are unknown samples, and etc. Using this additional data, the software then quantifies the unknown samples.

In another embodiment of the invention a kit containing at least two components is provided. The kit consists of a signal component which is a report element modified with a linker or tag (e.g. biotin), capable of linking or attaching to an affinity element (e.g. streptavidin). Alternatively, the signal component is a conjugate of a report element and a recognition element (e.g. antibody). The report element includes but not limited in a fluorescent dye labeled enzyme. Also included in the kit is the detection reagent. The detection reagent is a luminescence substrate for the enzyme that is corresponding to BRET process.

The kit is designed to run assays on equipment that includes a device to detect luminescence with spatial resolution along with the proper filter for the detection of the BRET process.

### EXAMPLES

### Example 1

### Conjugation of Alkaline Phosphatase with Fluorescent Dye

Alkaline phosphatase was conjugated to a fluorescent dye as follows: Dialyzed alkaline phosphatase was dissolved into PBS pH7.2. 200 μg of enzyme at 2.75 mg/ml was used for conjugation followed by the addition of one tenth volume of 1M NaHCO₃ at pH 9.0. To the mixture 2.1 μl of AF555, an amine reactive fluorescent dye, at concentration of 10 mg/ml in DMSO was added and the entire mixture was vortexed and then incubated at room temperature for one hour. The conjugated alkaline phosphatase-AF555 was separated from the reaction mixture using a P-30 (Bio-Rad) straw column previously equilibrated with PBS at pH 7.2.

### Example 2

### Spatial Resolution Test

The spatial resolution of the alkaline phosphatase-AF555 conjugate was tested as follows: 0.2 μl of a 100 nM solution of alkaline phosphatase-AF555 conjugate was spotted on a 1 cm diameter nitrocellulose membrane. Non-specific reactions were blocked by treating the membrane with 1% BSA in TBS at pH 8.5 for 5 minutes. The membrane was dried on a paper towel and placed on a microscope slide. 50 μl of alk-phos substrate Atto-Glow 530 was added to the membrane and a slide cover was placed over the membrane. The reaction image was photographed using a cooled CCD detector both with and without the chroma filter HQ560LP.

### Example 3

### Construction of Luciferase Conjugates

Luciferase conjugates were prepared as follows: Luciferase genes were amplified and cloned into a T7 expression vector (pRSET A). The luciferase genes were expressed in a codon plus BL21 (DE3) E. coli strain and the His-tag proteins were isolated and purified through the use of a standard nickel resin column. The fluorescent dye NHS-ester was conjugated to luciferase to form the report elements. Antibody was activated via a reaction with nitrilotriacetic acid and EDC to form a chelate precursor. Nickel ion binds to the chelate precursor to form a nickel-labeled antibody. Report element from (a) link to the antibody from (b) through the interaction of His-tag and nickel ion. This conjugate was used for the detection of a bio-agent through the mechanism of BRET.

### Example 4

### Light Differentiation

The light differentiation assay was performed as follows: The two luciferase conjugates were spotted on a nitrocellulose membrane. The substrate Chroma-Luc was added to generate light signals. The resulting light signals were recorded using a CCCD camera. The light intensity of each component of a mixed color sample can be determined by measuring the total light emitted by the mixed sample, filtered and unfiltered, and/or alternatively with multiple filters. Equations can then be written to separate the light contributions of the separate samples.

1. A method for the detection of bio-agents in a biological sample using bioluminescence resonance energy transfer (BRET) assay comprising:
(a) obtaining a biosample;
(b) contacting the biosample with a report element;
(c) allowing the report element to capture its target bio-agent;
(d) adding substrate for the report element;
(e) generating a light signal through BRET;
(e) detecting the light signal with a sensor.

2. The method according to claim 1, wherein the report element comprises a conjugate of a redox enzyme and a fluorescent element.

3. The method according to claim 2, wherein the redox enzyme is selected from the group consisting of alkaline phosphatase, luciferase, horse radish peroxidase and other redox enzymes.

4. The method according to claim 2, wherein the redox enzyme is alkaline phosphatase.

5. The method according to claim 2, wherein the fluorescent element is selected from the group consisting of fluorescent dyes, fluorescent proteins, quantum dots and other fluorescent elements.

6. The method according to claim 2, wherein the fluorescent element is directly linked to the redox enzyme.

7. The method according to claim 2, wherein the fluorescent element is indirectly linked to the redox enzyme.

8. A method for the comparison of bioagent levels in biological samples using a BRET assay comprising:
(a) obtaining two different biological samples;
(b) contacting a pair of report elements with both samples to capture targets;
(c) contacting the report elements with substrate;
(d) generating two light signals through BRET;
(e) detecting both light signals.

9. The method according to claim 8, wherein the report element comprises a pair of fluorescent elements conjugated to a redox enzyme.

10. The method according to claim 8, wherein the report element comprises a pair of homologous redox enzymes.

11. The method according to claim 10, wherein the pair of homologous redox enzymes comprise Luciferase-red and Luciferase-green.

12. The method according to claim 8, wherein the two report elements are linked to two anti-tag antibodies.

13. The method according to claim 8, wherein two tags are used to label bioagents from two samples.

14. The method according to claim 8, wherein specific recognition elements are used to capture target from two samples.

15. The method according to claim 8, wherein the interaction of the tags and their antibodies generates two light signals when substrate is present.

16. The method according to claim 14, wherein the recognition elements are selected from the group consisting of antibodies, receptors, ligands, nucleic acids and other biomolecules.

17. A method of signal amplification using BRET assay comprising:
(a) a recognition element;
(b) a signal molecule;
(c) peptide nucleic acids immobilized on a detection surface;
(d) a report element;
(e) generation of a light signal through BRET;
(f) detection of the light signal.

18. The method according to claim 17, wherein the recognition element comprises an oligonucleotide.

19. The method according to claim 18, wherein the oligonucleotide represents the recognition element specific target.

20. The method according to claim 19, wherein the oligonucleotide sequence comprises a tandem repeat.

21. The method according to claim 20 wherein the oligonucleotide sequence hybridizes to biotin labeled complimentary repeat.

22. The method according to claim 21, wherein the hybridized biotin labeled complimentary serves a signal molecule.

23. The method according to claim 20, wherein the complimentary repeats are released from the oligonucleotide when the recognition unit binds to the target.

24. The method according to claim 20, wherein the tandem repeat is bound by a conjugate of a streptavidin-report element.

25. The method according to claim 17, wherein the recognition element is selected from the group consisting of antibodies, receptors, ligands, and nucleic acids.

26. The method according to claim 17, wherein the optical sensor is selected from the group consisting of CCD based detectors, CMOS based detectors, luminometers, and light sensitive film.

27. A kit for use a BRET assay consisting of at least a signal component and detection element.

28. The kit according to claim 27, wherein the signal element comprises a report element modified with a linker capable of attaching an affinity element.

29. The kit according to claim 28, wherein the linker is biotin.

30. The kit according to claim 28, wherein the affinity element is streptavin.

31. The kit according to claim 27, wherein the signal component is a conjugate of a report element and a recognition element.

32. The kit according to claim 31, wherein the recognition element is selected from the group consisting of antibodies, ligands, receptors and nucleic acids.

33. The kit according to claim 31, wherein the report element is selected from the group consisting of fluorescent dye labeled enzyme, fluorescent protein labeled enzyme and quantum dot labeled enzyme.

34. The kit according to claim 27, wherein the detection element is a luminescence substrate.
